(19) United States
(12) Patent Application Publication
(10) Pub. No.: US 2022/0257307 A1
(43) Pub. Date: Aug. 18, 2022

(54) RESECTOSCOPE, ELECTRODE INSTRUMENT FOR A RESECTOSCOPE, AND GUIDE ELEMENT FOR AN ELECTRODE INSTRUMENT

(71) Applicant: Olympus Winter & Ibe GmbH, Hamburg (DE)

(72) Inventors: Christian BROCKMANN, Hollenstedt (DE); Christoph KNOPF, Stockelsdorf (DE); Andreas OFFT, Reinbek (DE); Hannes MIERSCH, Hamburg (DE)

(73) Assignee: Olympus Winter & Ibe GmbH, Hamburg (DE)

(21) Appl. No.: 17/665,669

(22) Filed: Feb. 7, 2022

(30) Foreign Application Priority Data
Feb. 5, 2021 (DE) 10 2021 102 735.7

(51) Int. Cl. A61B 18/14

(52) U.S. Cl. CPC A61B 18/149; A61B 2018/1465; A61B 2218/003

(57) ABSTRACT

A surgical radiofrequency instruments such as resectoscopes, for example, are used for removing or manipulating body tissue. A particular disadvantage of known instruments has been that the arrangement of an electrode instrument in an inner shaft can influence a flow behavior of an irrigation liquid. The invention makes available an electrode instrument by which the irrigation liquid is not disturbed. This is achieved by the fact that an electrode instrument has two electrode carriers which are arranged parallel to each other and are connected to each other by at least one guide element, and this at least one guide element extends, at least in part, convexly away from a longitudinal axis.

The invention relates to an electrode instrument for a resectoscope according to the preamble of claim 1. The invention further relates to a guide element according to claim 11 and to a resectoscope according to claim 17.

Surgical radiofrequency instruments such as resectoscopes, for example, are used for removing or manipulating body tissue. Typical uses are those in urology. One example that may be mentioned is resection of the prostate. A radiofrequency tool used in a resectoscope can be an electrode or RF electrode, which is attached to a radiofrequency generator, the generator being able to be activated and deactivated with a switch by an operator. The radiofrequency current has the effect that a plasma forms on the electrode. On account of the interaction of the plasma with the tissue, RF electrodes are particularly well suited for manipulation of the tissue with pinpoint accuracy.

For manipulating the tissue, the electrode, depending on the application, can be embodied as a cutting loop or as a button electrode, but also as a needle, roller, band, etc., and, with the radiofrequency voltage switched on, can be guided very easily and almost without resistance through the body tissue that is to be removed, this on account of the high temperature of the plasma.

The electrode is latched releasably to a working element of the resectoscope via an electrode instrument. During the treatment of the body tissue, the electrode instrument is moved with the electrode along a longitudinal direction of the resectoscope. Depending on whether the resectoscope is an active or passive resectoscope, the working element or the carriage is connected, by a compression spring or a tension spring, to a main body that has a gripping unit.

Known electrode instruments are guided in a tubular shaft, in particular an inner shaft, of the resectoscope, together with an optical unit which may be in the form of a light guide or rod lens system. This shaft extends from the proximal end to the distal end of the resectoscope and for treatment purposes is guided into the body to be treated, optionally together with an outer shaft. The electrode instrument is movably coupled to the optical unit inside the inner shaft. This can be done, for example, via a guide tube or a guide plate, which at least partially encloses the optical unit.

A particular disadvantage of this known system has been that the arrangement of the electrode instrument and of the optical unit in the inner shaft influences the flow behavior of an irrigation liquid. During the manipulation of the tissue, the view of the operator can be clouded by developing gas bubbles and by bleeding. To counteract this, the region in front of the distal end of the resectoscope is flushed clear by the irrigation liquid. The liquid is for this purpose conveyed through the inner shaft of the resectoscope and emerges at the distal end. For a clear view, it is essential that the emerging irrigation liquid has as far as possible a laminar behavior. An optimized flow can be achieved in particular if the flow of the irrigation liquid inside the shaft is not disturbed. However, such disturbance can be caused by the arrangement of the electrode instrument around the optical unit within the restricted interior of the inner shaft. The liquid flowing through the shaft impinges on the electrode instrument and/or the holders and starts swirling. This swirling of the irrigation liquid leads to a turbulent flow, which is particularly disadvantageous for the operator's view.

A further disadvantage of known systems is that the guide plate, particularly during assembly of the electrode instrument, becomes wedged with other components of the instrument. Since end faces of the guide plate and also end faces of other components are formed perpendicularly with respect to a longitudinal axis of the surgical instrument, this can lead to them becoming caught, which can result in an incorrect assembly of the electrode instrument, an incorrect function, or even damage to the components.

The object of the invention is to make available an electrode instrument, a guide plate and a resectoscope by which the problems mentioned above are solved.

This object is achieved by the features of claim 1. Accordingly, provision is made that an electrode instrument with an electrode has two portions, arranged parallel to each other and spaced apart from each other, of a respective electrode carrier, which portions are oriented at least substantially parallel to a longitudinal axis of the electrode instrument. An essential feature of the invention is that the two portions are connected to each other by at least one guide element, and this at least one guide element, at least in part, extends convexly away from the longitudinal axis. This design allows the electrode instrument to be produced in a particularly compact form, such that a cross section occupied by the electrode instrument in the resectoscope is particularly small. Moreover, a constant cross section has a particularly advantageous effect as regards the use of the instrument. Thus, the irrigation liquid flowing through the resectoscope is only minimally disturbed or not disturbed at all, such that a laminar flow is generated at the distal end and in the interior of the resectoscope, and this laminar flow can permit particularly advantageous treatment conditions. Moreover, the two portions of the electrode carrier, or the entire electrode instrument, are stabilized by the at least one guide element. Such stabilization is particularly advantageous if the cross section of the portions, or of the electrode casing tubes, is very small and the length of the stated component is very large. By means of the portions being mutually fixed by the guide element, particularly thin and particularly long electrodes can also be used easily and with pinpoint accuracy.

In the present invention, provision can be made in particular that the two portions are designed as electrode casing tubes which extend parallel to each other from the distal end to the proximal end of the electrode instrument, or that the two portions have a fork-like design and converge in the direction of the proximal end of the electrode instrument to form an electrode shaft. As a result of this parallel course of the casing tubes, the electrode instrument can be particularly advantageously oriented parallel to a shaft, for example an inner shaft, of the resectoscope.

Provision can be made, according to the invention, that the electrode instrument with its at least one guide element is not placed as before on the optical unit inside the inner shaft, but instead on the inner shaft of the resectoscope and is mounted so as to be movable along the longitudinal axis. By virtue of this special design of the electrode instrument, the portions of the electrode carriers, or the electrode casing tubes, can be relocated out of the inner shaft and onto the inner shaft. Thus, the components of the electrode instrument can no longer disturb the irrigation liquid flowing into the inner shaft. Rather, the liquid can now pass unimpeded through the resectoscope and establish the advantageous laminar flow. Furthermore, it is conceivable that the cross section of the inner shaft can be reduced as a result of this relocation of the electrode instrument.

Provision is preferably made that two or more guide elements are arranged on the two electrode casing tubes. By increasing the number of guide elements, the stability and also the handling of the overall electrode appliance can be improved.

In a further advantageous illustrative embodiment of the invention, provision can be made that the guide element has, perpendicular to the longitudinal axis, an arc-like cross section, preferably a circular-segment-like cross section. This cross section can extend over an angle range of 30°-270°, in particular 30°-180°, preferably 45°-120°, or a value lying between these values. By virtue of this circular-segment-like cross section, the electrode instrument can be placed particularly easily and reliably onto an outer wall of the inner shaft. By virtue of this advantageous embodiment of the guide elements, the electrode instrument is particularly easily movable over the inner shaft in the direction of the longitudinal axis.

Provision can also be made that the cross section of the guide element corresponds, with its shape or its radius, to the shape or the radius of a component, in particular an inner shaft or an optical unit, of the resectoscope, such that the guide element can be placed, in particular clamped, on and/or under this component. The at least one guide element is thus designed in such a way that the electrode instrument can be moved along the inner shaft in a predetermined manner.

Furthermore, it is conceivable according to the invention that an undercut for a latching connection to the inner shaft or to the optical unit is formed from the guide element and the electrode casing tubes. By an elastic deformation of the guide element, the electrode instrument can nonetheless be clipped over the inner shaft or the optical unit and is held in the direction of joining together.

In a further illustrative embodiment, provision can be made that the guide element can be joined around or onto a component, preferably an inner shaft or an optical unit, of the resectoscope, such that the electrode instrument is movable relative to the component along the longitudinal axis.

Preferably, it is also conceivable that the guide element has, at two opposite sides, circular-segment-like receptacles which correspond to the shape of the portions or of the casing tubes, wherein these receptacles engage at least partially around the portions or the casing tubes from the outside or inside. By way of these receptacles, the guide elements can be fastened particularly easily and flexibly onto the portions. It is conceivable here that the receptacles are fastened to the portions both from the outside and from the inside. In addition, it is likewise conceivable that the guide elements are not fixed to the portions releasably but instead permanently via the receptacles. This makes it possible to prevent a situation where, in the event of considerable force being applied parallel or transverse to the longitudinal axis, the guide elements are displaced and the stability of the electrode instrument is thereby reduced.

In a particularly preferred illustrative embodiment of the present invention, provision is made that a distance between two opposite sides of the guide element measures 3 mm to 8 mm. This spacing of the opposite sides allows the electrode instrument to be fixed easily and reliably around the inner shaft. The plate-like guide element can thus be clipped onto the shaft, for example. A suitable material for the guide element is a metal or a reversibly deformable plastic. On the one hand, such materials have the appropriate properties for the flexible connection of the electrode instrument to the inner shaft, and, on the other hand, they are also particularly easy to clean. A suitable shape for the guide element is a convexly shaped rectangle or a cuboid. However, it is also conceivable that the guide elements have another kind of shape. Ultimately, the guide element has to be designed in such a way that it can guide the electrode instrument along the inner shaft.

A guide element for achieving the stated object has the features as per claim 11. Accordingly, a guide element is provided for an electrode instrument of a resectoscope as claimed in one of claims 1-10. This guide element is characterized in that it has an arc-like cross section, preferably a circular-segment-like cross section, which corresponds, with its shape or its radius, to the shape or the radius of a component, preferably an inner shaft or an optical unit, of the resectoscope, such that the guide element can be placed, in particular clamped, on and/or under this component.

In a preferred illustrative embodiment of the guide element, provision can be made that the latter has, at two opposite sides, circular-segment-like receptacles which are oriented parallel to each other and which are guided outward or inward, and wherein a distance between the two mutually opposite sides or the receptacles of the guide element measures 3 mm to 8 mm. These receptacles serve to fasten the guide element around two parallel components of the electrode instrument from the outside or to fasten it from the inside outward onto said components. This design of the guide element allows it to be coupled to the electrode instrument in a simple and flexible manner.

In a further possible embodiment of the invention, provision can be made that at least one end face, formed in particular with an arc-like or circular-segment-like cross section, is perpendicular to a longitudinal axis of the guide element, or that the at least one end face has a recess that extends in the direction of an opposite end face of the guide element. During assembly of the guide element or the electrode instrument, this recess can prevent same from becoming caught or wedged on a component of the electrode instrument or of the resectoscope. By means of this recess, the contact area is reduced between an inner face of the guide element and the component and/or the inner shaft of, the resectoscope. As a result of this reduction of the contact area, the risk of wedging or catching is also reduced.

It is furthermore conceivable that the aforementioned recess together with formed in the shape of an arc of a circle or a polygon, in particular like a triangle. In particular, giving the recess the shape of an arc of a circle simplifies the assembly of the electrode instrument, since the clearance for movement is increased by the round recess. It is conceivable here that the recess has a radius of curvature similar or identical to that of the inner shaft.

It is furthermore conceivable that a tangent to the recess of the longitudinal axis of the guide element of the, preferably along the entire length of the end face, enclose an angle α not equal to 90°. The risk of wedging can be reduced particularly efficiently by this configuration.

Preferably, according to the invention, provision can also be made that the guide element is symmetrical, wherein the two side edges and the two end faces with the recesses are symmetrical to one another. This symmetry simplifies the assembly of the components and also the method of production of the guide element.

Finally, a resectoscope as per claim 17 also achieves the object mentioned at the outset. This resectoscope has an electrode instrument as per claims 1-10. Furthermore, the resectoscope has an inner shaft in which an optical unit is mounted, wherein the electrode instrument can be guided on the inner shaft with at least one guide element as per at least one of claims 11 through 16.

Moreover, provision is preferably made that a longitudinal axis of the electrode instrument is arranged above or below a longitudinal axis of the inner shaft, or that a longitudinal axis of the electrode instrument and a longitudinal axis of the inner shaft lie in one plane. It is conceivable here that the guide element is arranged above the longitudinal axis of the electrode instrument and open toward the bottom or that the guide element is arranged below the longitudinal axis of the electrode instrument and open toward the top. By virtue of this arrangement of the electrode instrument, the individual components of the resectoscope can be oriented and positioned relative to one another in a way that particularly saves space, specifically without the flow behavior of the irrigation liquid being disturbed.

A preferred illustrative embodiment of the invention is described in more detail below with reference to the drawing, in which:

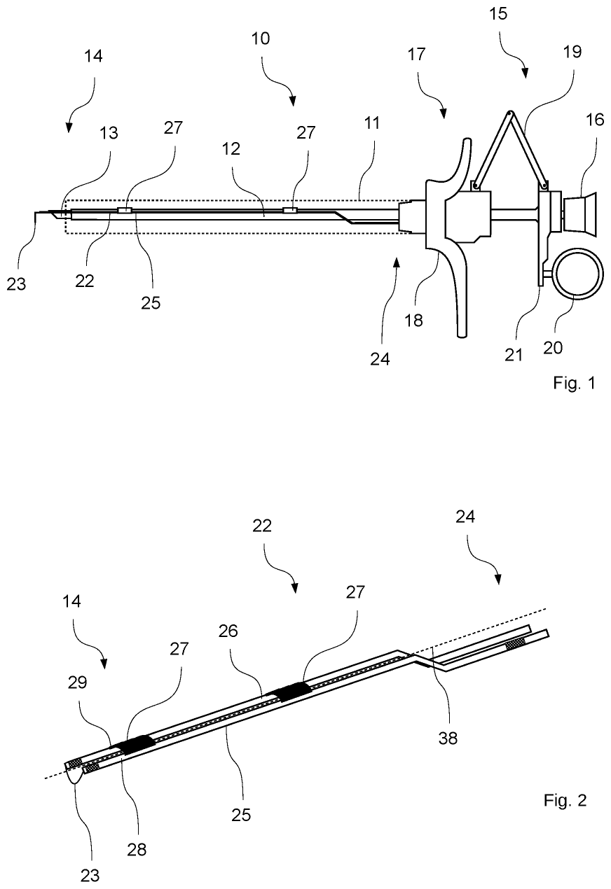

FIG. 1 shows a schematic view of a resectoscope,

FIG. 2 shows a schematic perspective view of an electrode instrument,

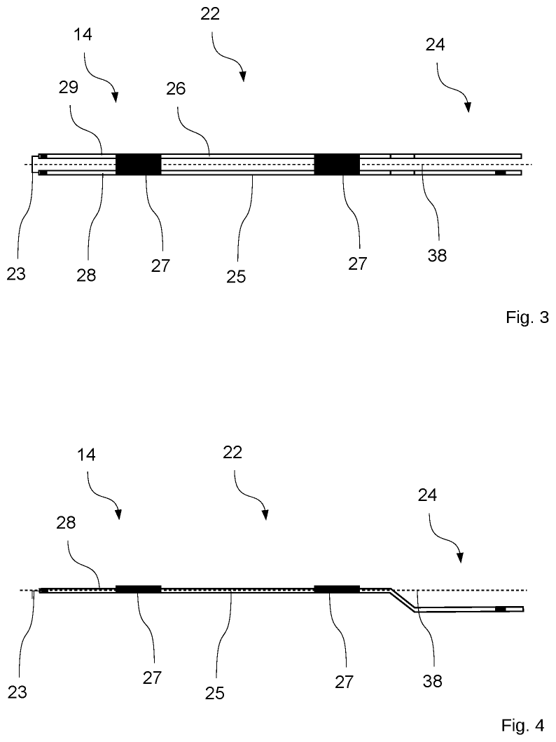

FIG. 3 shows a view of the electrode instrument according to FIG. 2,

FIG. 4 shows a side view of the electrode instrument according to FIG. 2,

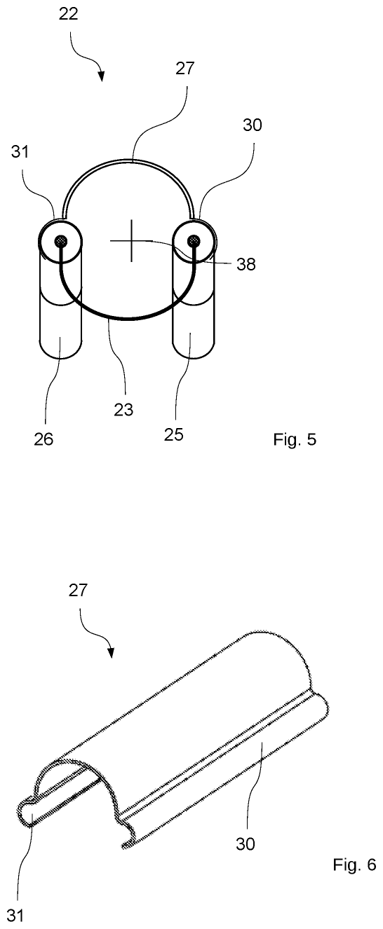

FIG. 5 shows a view of the distal end of the electrode instrument according to FIG. 2,

FIG. 6 shows a perspective view of a guide element,

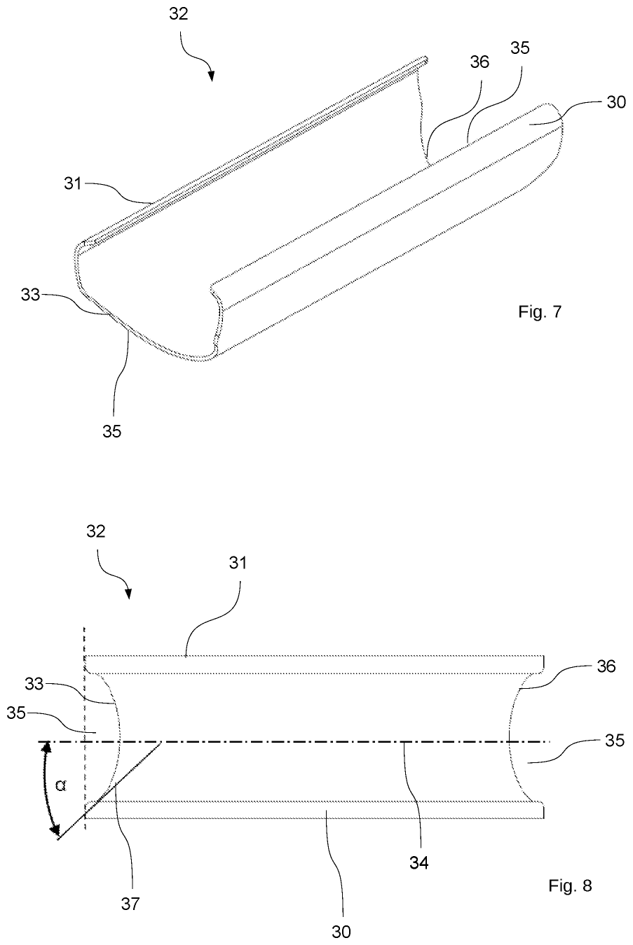

FIG. 7 shows a perspective view of a further illustrative embodiment of a guide element,

FIG. 8 shows a view of the guide element according to FIG. 7,

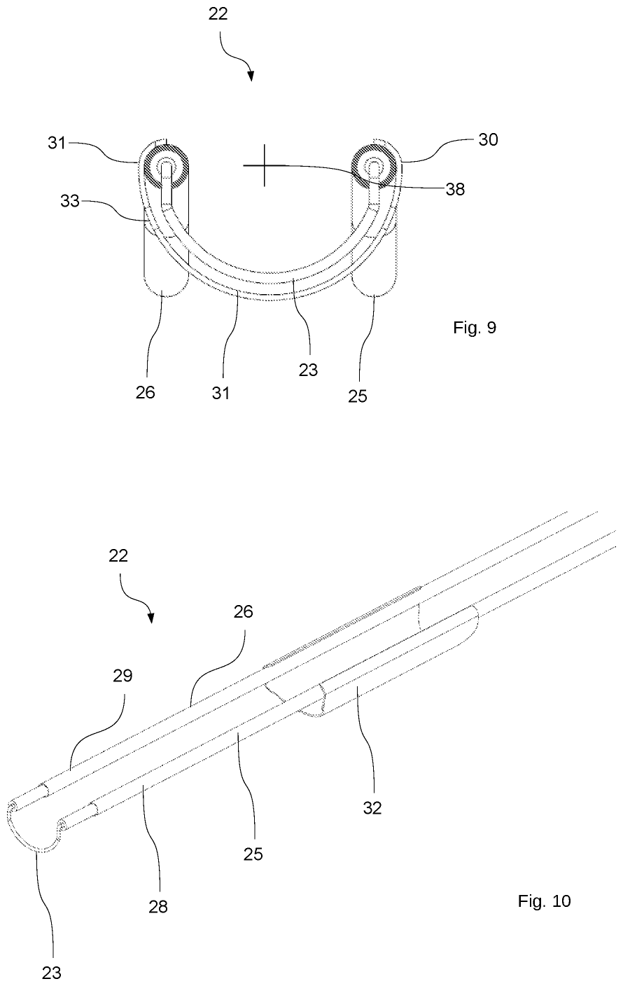

FIG. 9 shows a view of the distal end of the electrode instrument according to FIG. 7, and

FIG. 10 shows a schematic perspective view of an electrode instrument with the guide element according to FIG. 7.

FIG. 1 shows a possible illustrative embodiment of a resectoscope 10. In this resectoscope 10, an outer shaft 11, only symbolized here by dashed lines, is pushed over an inner shaft 12. The inner shaft 12 serves to receive and/or guide an optical unit 13, which extends from a distal end 14 to a proximal end 15 of the resectoscope 10. At the proximal end 15, an eyepiece 16 is available to allow a user to observe, through the optical unit 13, the region in which surgery is to be performed in front of the distal end 14.

An essential component of the resectoscope 10 is the working element 17. This working element 17 has a first gripping means 18, among other things, and is connected by a spring element 19 to a second gripping means 20 and to an optical plate 21.

Moreover, an electrode instrument 22 extends along the inner shaft 12 from the distal end 14 of the resectoscope 10 as far as the working element 17. The electrode instrument 22 shown in FIGS. 2 to 4 is just one possible illustrative embodiment. It is expressly noted that the invention described here is not intended to be limited to the form that is presented here. Instead, it is conceivable that the described invention can also be used in connection with electrode instruments of another form.

The electrode instrument 22 shown here can be latched with a proximal end 24 in the working element 17. In this way, on the one hand, the electrode instrument 22 can be easily uncoupled from the working element 17 or coupled to the working element 17 and, on the other hand, can move together with the working element 17 along the longitudinal axis of the resectoscope 10 in the distal or proximal direction.

At the distal end 14 of the electrode instrument 22, the latter has an electrode 23. Electrical energy is able to be applied to this electrode 23, or cutting electrode, by means of an RF generator (not shown) and serves to manipulate tissue. By applying an RF voltage to the electrode, a plasma forms around the electrode 23, here shown as a cutting loop. The organic tissue can be manipulated or cut by an axial forward and backward movement of the electrode instrument 22. In addition to the cutting loop shown in the figures, other electrode forms are also conceivable.

For manipulating human tissue with pinpoint accuracy, it is crucially important that the electrode 23 can be handled in a very precise manner. This precision handling is made difficult in particular by a lengthening of the electrode instrument 22 or by a reduction of the cross section of the instrument 22.

As can be seen in FIGS. 2 to 5, the electrode 23, by way of its ends, is mechanically fastened or able to be mechanically fastened to two electrode carriers 25 and 26 or to two electrode casing tubes. The electrode 23 and the electrode carriers 25 and 26 represent the essential constituent parts of the electrode instrument 22. In addition to the parallel arrangement (shown here) of the electrode carriers 25 and 26 or electrode casing tubes, it is also conceivable for the electrode carriers 25 and 26 to have a fork-like design and to converge in the direction of the proximal end 15 to form a shaft.

In addition to the mechanical connection, the electrode carriers 25 and 26 or the electrode casing tubes also serve to electrically contact the electrode 23. Provision is made that the electrode carriers 25 and 26, or electrode casing tubes, and also electrical leads within the carriers 25 and 26 serve as electrical lines or contacts.

In order to increase the stability of the electrode instrument 22, and the associated secure and precise handling thereof, the invention proposes that at least distal portions 28, 29 of the electrode carriers 25, 26 be connected to each other by a guide element 27. Furthermore, provision can be made, or it is conceivable, that the two electrode carriers 25, 26 are connected to each other by at least one further guide element 27.

This guide element 27 serves not only for the stabilizing of the whole electrode instrument 22, but also for the guiding along the inner shaft 12. According to the invention, the at least one guide element 27 is placed on a jacket surface of the inner shaft 12, the guide element 27 at least partially enclosing the inner shaft 12. Thus, in contrast to the prior art, the electrode instrument 22 is no longer mounted on the optical unit 13 inside the inner shaft 12, but instead outside the shaft 12. In this way, the interior of the inner shaft 12 is free for undisturbed flow of the irrigation liquid. This has a particularly advantageous effect on the flow behavior of the irrigation liquid in the region in front of the optical unit 13 outside the shaft 11. The generated laminar flow of the irrigation liquid provides the operator with optimal viewing of the treatment region. Equally, relocating the electrode instrument 22 to outside the inner shaft 12 permits a reduction of the diameter of the inner shaft 12. This reduction of the diameter of the inner shaft 12 has a positive impact on the overall design of the resectoscope 10, for example because the diameter of an outer shaft of the resectoscope 10 can thus also be reduced. Miniaturization of the instruments allows treatment to be performed in a way that is particularly gentle on the body.

According to the invention, provision can also be made that, for example as is shown in FIG. 1, the electrode instrument 22 is placed onto the inner shaft 12 via the guide elements 27 in such a way that it is arranged above a longitudinal axis of the inner shaft 12. It has been shown that this arrangement is particularly effective in saving space and equally permits precise positioning of the electrode 23 in front of the distal end of the inner shaft 12.

However, it is also conceivable that the guide element 32 can be fastened onto the electrode carriers 25, 26 from below, the electrode instrument 22 then being located underneath the longitudinal axis of the inner shaft 12 (FIGS. 7 to 10). This has the advantage that the shape of the guide element 32 is simpler, since the circular-segment-like receptacles 30, 31 can be smaller. The receptacles 30, 31 are moreover curved in the same direction, like the guide element 32 itself, which makes production easier.

Provision is preferably made that the free ends of the guide elements 27, 32 point in the same spatial direction. Thus, when welding the guide elements 27, 32 to the electrode carriers 25, 26, both sides can be welded one directly after the other, specifically without the components having to be turned for this purpose. As a result, the production method proves particularly efficient.

To ensure that the electrode instrument 22 can be moved particularly reliably along the inner shaft 12, the guide element 27 is convex or forms, in its cross section, a segment of a circle (FIGS. 5 and 6), the convex curvature facing away from the electrode carriers. The radius of curvature of the guide element corresponds here to the shape or the radius of curvature of the inner shaft 12. In this way, the guide element 27 or guide elements 27 join particularly advantageously to the outer face of the inner shaft 12.

In addition to the embodiment of the guide element 27 shown in FIGS. 5 and 6, it is also conceivable that the cross section of the guide element 27 describes a segment of a circle greater than 180°. A guide element 27 of this shape can be clamped or clipped onto the inner shaft 12. A guide element 27 of this shape can also be releasably connected to the inner shaft 12 from below (see above). It is accordingly conceivable that different embodiments of electrode instruments with the guide elements 27, 32 described here are positioned on the inner shaft 12 from above or from below.

In order to connect the guide element 27 reliably to the electrode carriers 25, 26, it has circular-segment-like receptacles 30, 31 at two opposite sides (FIGS. 5, 6 and 9). These receptacles 30, 31 close at least partially, preferably completely, around the tubular electrode carriers 25, 26. It is conceivable here that the receptacles 30, 31 are connected to the electrode carriers 25, 26 by adhesive bonding, welding or crimping, for example.

The illustrative embodiment of a guide element 27 shown in FIG. 6 is designed in such a way that it can be placed onto the two electrode carriers 25, 26 from above, and the receptacles 30, 31 engage around the electrode carriers 25, 26. This connection of the guide element 27 to the electrode carriers 25, 26 ensures that the electrode instrument 22 can be moved relative to the inner shaft 12 in a safe and well defined manner.

In a preferred illustrative embodiment, provision is made that the receptacles 30, 31 of the guide element 27 are at a distance of between 3 and 8 mm from each other. It is equally conceivable that this distance is greater or smaller. The guide element 27 is preferably made from a plastic or a metal, the wall thickness being 0.1 mm to 1.0 mm, preferably 0.1 mm to 0.3 mm. A wall thickness of greater than 2 mm is also conceivable.

A further illustrative embodiment of a guide element 32 according to the invention is shown in FIGS. 7 to 10. Just like the guide element 27, this illustrative embodiment also has elongate receptacles 30, 31 arranged parallel to a longitudinal axis 34. The guide element 32 is also connectable to the electrode instrument 22 via these receptacles 30, 31. The guide element 32 can be pushed over the inner shaft 12 both above and also below the longitudinal axis 38 of the electrode instrument 22. Exactly as described before for the guide element 27, the guide element 32 is also pushed over the electrode carriers 25, 26 are fixedly connected to these, for example by welding.

The guide element 32 shown in FIGS. 7 and 8 has a recess 35 at both end faces 33, 36. However, it is also conceivable that only one of the two end faces 33, 36 has such a recess 35. As can be seen in FIG. 8, the end faces 33, 36 are configured in an arc shape to form the recess 35. This arc is preferably configured in such a way that a tangent 37 to the end faces 33, 36 encloses, at almost every point of the end faces 33, 36, a non-90° angle α with the longitudinal axis 34. The angle α can be equal to 90° only at an imaginary intersection between the longitudinal axis 34 and the end faces 33, 36. In addition to the arc-shaped configuration of the recess 35 shown here, other shapes are also conceivable. Thus, provision can also be made that the end faces 33, 36 have the shape of a triangle.

By virtue of the arc-shaped configuration of the end faces 33, 36 and by virtue of the recesses 35, the mounting of the guide element 32 on the electrode instrument 22 and the mounting of the electrode instrument 22 with guide element 32 on the inner shaft 12 prove particularly advantageous. By virtue of the recess 35, the contact area between the guide element 32 and the inner shaft 12 is reduced, and therefore the danger of wedging or catching is likewise reduced.

List of reference signs

| | |
|---|---|
| 10 | resectoscope |
| 11 | shaft |
| 12 | inner shaft |
| 13 | optical unit |
| 14 | distal end |
| 15 | proximal end |
| 16 | eyepiece |
| 17 | working element |
| 18 | first gripping means |
| 19 | spring element |
| 20 | second gripping means |
| 21 | optical plate |
| 22 | electrode instrument |
| 23 | electrode |
| 24 | proximal end |
| 25 | electrode carrier |
| 26 | electrode carrier |
| 27 | guide element |
| 28 | portion |
| 29 | portion |
| 30 | receptacle |
| 31 | receptacle |
| 32 | guide element |
| 33 | end face |
| 34 | longitudinal axis |
| 35 | recess |
| 36 | end face |
| 37 | tangent |
| 38 | longitudinal axis of the electrode |
| | instrument |

1. An electrode instrument for a resectoscope with an electrode, the electrode being fastened to two distal ends of two tubular portions, spaced apart from each other and parallel to each other, of a respective electrode carrier, and the portions being oriented at least substantially parallel to a longitudinal axis of the electrode instrument, wherein at least one guide element is arranged on the electrode carriers, which guide element connects the two portions to each other, is convex at least in part, and extends away from the longitudinal axis.

2. The electrode instrument as claimed in claim 1, wherein the two portions are designed as electrode casing tubes which extend parallel to each other from the distal end to the proximal end of the electrode instrument, or in that the two portions have a fork-like design and converge in the direction of the proximal end of the electrode instrument to form an electrode shaft.

3. The electrode instrument as claimed in claim 2, wherein two or more guide elements are arranged on the two electrode casing tubes.

4. The electrode instrument as claimed in claim 1, wherein the guide element has, perpendicular to the longitudinal axis, an arc-like cross section.

5. The electrode instrument as claimed in claim 4, wherein the cross section corresponds, with its shape or its radius, to the shape or the radius of a component, of the resectoscope, such that the guide element can be placed, on and/or under this component.

6. The electrode instrument as claimed in claim 1, wherein an undercut for a latching connection to the inner shaft or the optical unit is formed from the guide element and the electrode casing tubes.

7. The electrode instrument as claimed in claim 1, wherein the guide element can be joined around or onto a component, of the resectoscope, such that the electrode instrument is movable relative to the component along the longitudinal axis.

8. The electrode instrument as claimed in claim 1, wherein the guide element has, at two opposite sides, circular-segment-like receptacles which correspond to the shape of the portions or the electrode casing tubes, wherein these receptacles engage at least partially around the portions or electrode casing tubes from the outside or inside.

9. The electrode instrument as claimed in claim 1, wherein the guide element can be arranged on the portions above or below the longitudinal axis of the electrode instrument, the guide element being arranged above the longitudinal axis of the electrode instrument and being open toward the bottom, or the guide element being arranged below the longitudinal axis of the electrode instrument and being open toward the top.

10. The electrode instrument as claimed in claim 1, wherein a distance between two opposite sides of the guide element measures 3 mm to 8 mm.

11. A guide element for an electrode instrument of a resectoscope as claimed in claim 1, wherein an arc-like cross section, which corresponds, with its shape or its radius, to the shape or the radius of a component, of the resectoscope, such that the guide element can be placed on and/or under this component.

12. The guide element for an electrode instrument as claimed in claim 11, wherein the guide element has, at two opposite sides, circular-segment-like receptacles which are oriented parallel to each other and which are guided outward or inward, and wherein a distance between the two opposite sides or the receptacles of the guide element measures 3 mm to 8 mm.

13. The guide element for an electrode instrument as claimed in claim 11, wherein at least one end face, formed with an arc-like or circular-segment-like cross section, is perpendicular to a longitudinal axis of the guide element, or in that the at least one end face has a recess that extends in the direction of an opposite end face.

14. The guide element for an electrode instrument as claimed in claim 13, wherein the recess is shaped like an arc of a circle, or like a polygon.

15. The guide element for an electrode instrument as claimed in claim 13, wherein a tangent to the recess encloses, with the longitudinal axis along the entire length of the end face, an angle α not equal to 90°.

16. The guide element for an electrode instrument as claimed in claim 11, wherein the guide element is symmetrical, wherein the two side edges and the two end faces with the recesses are symmetrical to one another.

17. A resectoscope with an electrode instrument as claimed in claim 1 and with an inner shaft in which an optical unit is mounted, wherein the electrode instrument can be guided on the inner shaft with at least one guide element.

18. The resectoscope as claimed in claim 17, wherein a longitudinal axis of the electrode instrument lies above or below a longitudinal axis of the inner shaft, or in that a longitudinal axis of the electrode instrument and a longitudinal axis of the inner shaft lie in one plane.
